(19) United States
(12) Patent Application Publication
(10) Pub. No.: US 2024/0148308 A1
(43) Pub. Date: May 9, 2024

(54) SLEEP APNEA RISK BASED ON BRADYCARDIA BURDEN

(71) Applicant: Cardiac Pacemakers, Inc., St. Paul, MN (US)

(72) Inventors: David L. Perschbacher, Blaine, MN (US); Keith Joseph Mattson, Lino Lakes, MN (US); Scott R. Vanderlinde, Plymouth, MN (US); Abhijit Rajan, Shoreview, MN (US)

(21) Appl. No.: 18/501,257

(22) Filed: Nov. 3, 2023

Related U.S. Application Data
(60) Provisional application No. 63/422,309, filed on Nov. 3, 2022.

(51) Int. Cl. A61B 5/363; A61B 5/00

(52) U.S. Cl. CPC A61B 5/363; A61B 5/4818; A61B 5/7275; A61B 5/7282; A61B 5/7292; A61B 5/746

(57) ABSTRACT

Systems and methods for monitoring a subject for sleep apnea risk based on a bradycardia burden are disclosed. A sleep apnea monitoring system comprises a risk stratification circuit configured to detect bradycardia using cardiac information of the subject during a monitoring period, determine a bradycardia burden representing accumulated time or relative time of the subject being in the detected bradycardia over the monitoring period, and determine an apnea risk based on the bradycardia burden. An output unit can provide the apnea risk to a user, or to a process executed by an apnea detector to perform a confirmatory apnea detection.

### CLAIM OF PRIORITY

This application claims the benefit of U.S. Provisional Application No. 63/422,309, filed on Nov. 3, 2022, which is hereby incorporated by reference in its entirety.

### TECHNICAL FIELD

This document relates generally to medical devices, and more particularly, to systems, devices and methods determining a sleep apnea risk in a patient based on bradycardia burden.

### BACKGROUND

Sleep apnea involves a brief cessation of breathing during sleep. The most common type of sleep apnea is central sleep apnea. Central sleep apnea (CSA) is associated with the failure of the body to automatically initiate and control a respiratory cycle at the proper time. CSA typically causes cessation of substantially all respiratory effort during sleep. CSA may be developed after a heart attack, and is usually a contributing factor to heart failure and other cardiopulmonary disorders. Obstructive sleep apnea (OSA) is associated with a blockage of the airway, and is generally characterized by repetitive pauses in breathing during sleep due to upper airway obstruction or collapse. OSA is commonly found in overweight people who snore or have oversized necks. When awake, muscle tone keeps the throat open. When asleep, the airway of the neck narrows and closes. The person struggles to breathe against the collapsed throat as if choking. As the patient wakes up, the muscles of the throat open the airway. Many patients with congestive heart failure (CHF) suffer from obstructive sleep apnea.

Implantable medical devices (IMDs) have been used for monitoring patient health condition or disease states and delivering therapies. For example, implantable cardioverter-defibrillators may be used to monitor for certain abnormal heart rhythms, such as bradycardia or tachycardia, and to deliver electrical energy to the heart to correct the abnormal rhythms. Some IMDs may be used to monitor for chronic worsening of cardiac hemodynamic performance, such as due to congestive heart failure (CHF), and to provide cardiac stimulation therapies, including cardiac resynchronization therapy (CRT) to correct cardiac dyssynchrony within a ventricle or between ventricles.

Sleep apnea is one of common comorbidities in patients with various heart conditions. Certain cardiac diseases such as CHF can be worsened by excessive stress during apnea. Early and accurate identification of patients at elevated risk of sleep apnea can be clinically important for assessing patient cardiac function and help prevent or slow worsening of certain heart conditions.

### OVERVIEW

Sleep apnea detection generally involves direct measurement of respiration parameters such as respiration rate, tidal volume, respiration flow, or a respiration pattern, or indirect respiration measurement such as transthoracic impedance, acoustic signals indicative of respiration, or other respiration surrogates. Although effective in detecting full-blown sleep apnea, direct or indirect respiration-based approaches may not be sensitive enough to provide an earlier indication of potential apnea events or be used for risk stratification of sleep apnea. Sleep study is the clinical standard for diagnosing sleep apnea and typically performed in a sleep lab. However, a sleep study can be less convenient and/or less comfortable for some patients. Patients may also be constrained by accessibility, scheduling, and cost associated with a sleep study.

Certain cardiac arrhythmias, particularly bradycardia episodes or cardiac pause events, are associated with sleep apnea. Cardiac pause (or sinus pause) refers to an absence of electrical activity in the heart for an extended period of time. This may be attributed to sinus arrest, a condition where the sinoatrial node of the heart transiently ceases to generate the electrical impulses that normally stimulate the myocardial tissues to contract and thus the heart to beat. Sinus pause may cause severe lightheadedness, dizziness, near syncope, or a syncopal or passing-out episode. Nocturnal bradycardia, including nocturnal sinus pause, can occur in up to 50% of patients with OSA. On the other hand, apnea treatment (e.g., CPAP treatment) has been shown to reduce nocturnal bradycardia event rate.

The present inventors have recognized an unmet need for apparatus and techniques to more conveniently and effectively identify patients at an elevated risk of sleep apnea with adequate accuracy. The present document discusses, among other things, systems, devices, and methods for monitoring a subject for risk of sleep apnea based on bradycardia burden. In accordance with an embodiment, a sleep apnea monitoring system comprises a risk stratification circuit configured to detect bradycardia using cardiac information of the subject during a monitoring period, and determine a bradycardia burden based on the detected bradycardia. The bradycardia burden represents accumulated time or relative time of the subject being in the detected bradycardia during the monitoring period. The sleep apnea risk stratification circuit can determine an apnea risk based on the determined bradycardia burden. The apnea risk can be presented to a user, or provided to a process such as confirmatory detection of sleep apnea executed by a respiration-based apnea detector.

Example 1 is a system for monitoring a subject for risk of sleep apnea. The system comprises: a sleep apnea risk stratification circuit configured to: receive cardiac information of the subject; detect bradycardia from the received cardiac information during a monitoring period; determine a bradycardia burden based on the detected bradycardia, the bradycardia burden representing accumulated time or relative time of the subject being in the bradycardia during the monitoring period; and determine an apnea risk based on the bradycardia burden; and an output unit configured to provide the determined apnea risk to a user or a process.

In Example 2, the subject matter of Example 1 optionally includes an ambulatory medical device configured to collect the cardiac information including a surface or subcutaneous electrocardiogram from the subject, the ambulatory medical device including the sleep apnea risk stratification circuit.

In Example 3, the subject matter of any one or more of Examples 1-2 optionally include, wherein to detect the bradycardia includes to determine a duration of a bradycardia episode, the bradycardia episode including one or more of a sinus pause episode, a sinus bradycardia episode, or an atrioventricular disturbance or atrioventricular block episode.

In Example 4, the subject matter of any one or more of Examples 1-3 optionally include the sleep apnea risk stratification circuit that can be configured to determine the bradycardia burden based on the bradycardia detected during a nighttime monitoring period.

In Example 5, the subject matter of any one or more of Examples 1˜4 optionally include the sleep apnea risk stratification circuit that can be configured to determine the bradycardia burden based on the bradycardia detected during a time period when the subject is asleep.

In Example 6, the subject matter of Example 5 optionally includes a sleep detector configured to detect a sleep or awake state of the subject, wherein the sleep apnea risk stratification circuit is configured to determine the bradycardia burden based on the bradycardia detected when the detected sleep or awake state indicates that the subject is asleep.

In Example 7, the subject matter of any one or more of Examples 1-6 optionally include the sleep apnea risk stratification circuit that can be configured to detect one or more bradycardia episodes each satisfying a rate or duration requirement, and to determine the bradycardia burden based on a count of the detected one or more bradycardia episodes that satisfy the rate or duration requirement during the monitoring period.

In Example 8, the subject matter of Example 7 optionally includes the one or more bradycardia episodes that can include one or more sinus pause episodes each lasting at least a threshold duration.

In Example 9, the subject matter of any one or more of Examples 7-8 optionally include the one or more bradycardia episodes that can include one or more sinus bradycardia episodes each having an average heart rate below a threshold heart rate.

In Example 10, the subject matter of any one or more of Examples 7-9 optionally include the one or more bradycardia episodes that can include one or more episodes of atrioventricular disturbance or atrioventricular block each lasting at least a threshold duration.

In Example 11, the subject matter of any one or more of Examples 1-10 optionally include, wherein to determine the apnea risk includes to determine a numerical risk score directly proportional to the bradycardia burden.

In Example 12, the subject matter of any one or more of Examples 1-11 optionally include the sleep apnea risk stratification circuit that can be configured to trend the bradycardia burden over time, and to determine the apnea risk based on the trended bradycardia burden.

In Example 13, the subject matter of any one or more of Examples 1-12 optionally include the output unit that can be configured to generate an alert to the user in response to the determined apnea risk exceeding a risk threshold.

In Example 14, the subject matter of any one or more of Examples 1-13 optionally include the output unit that can be configured to generate a recommendation of apnea evaluation or treatment in response to the determined apnea risk exceeding a risk threshold.

In Example 15, the subject matter of any one or more of Examples 1-14 optionally include the output unit that can be configured to generate a control signal to a respiration-based apnea detector to trigger sleep apnea detection in response to the determined apnea risk exceeding a risk threshold.

Example 16 is a method of monitoring a subject for risk of sleep apnea, the method comprising: receiving cardiac information of the subject; detecting bradycardia from the received cardiac information during a monitoring period; determining a bradycardia burden based on the detected bradycardia, the bradycardia burden representing accumulated time or relative time of the subject being in the bradycardia during a monitoring period; and determining an apnea risk based on the bradycardia burden using a sleep apnea risk stratification circuit; and providing the determined apnea risk to a user or a process.

In Example 17, the subject matter of Example 16 optionally includes, wherein determining the bradycardia burden is based on the bradycardia detected during a nighttime monitoring period.

In Example 18, the subject matter of any one or more of Examples 16-17 optionally include, wherein determining the bradycardia burden is based on the bradycardia detected during a monitoring period when the subject is asleep.

In Example 19, the subject matter of any one or more of Examples 16-18 optionally include, wherein: detecting the bradycardia incudes detecting one or more bradycardia episodes each satisfying a rate or duration requirement, determining the bradycardia burden is based on a count of the detected one or more bradycardia episodes during the monitoring period.

In Example 20, the subject matter of Example 19 optionally includes, wherein the one or more bradycardia episodes include one or more sinus pause episodes each lasting at least a threshold duration.

In Example 21, the subject matter of any one or more of Examples 19-20 optionally include, wherein the one or more bradycardia episodes include one or more sinus bradycardia episodes each having an average heart rate below a threshold heart rate.

In Example 22, the subject matter of any one or more of Examples 19-21 optionally include generating a bradycardia burden trend over time, wherein determining the apnea risk is based on the bradycardia burden trend.

In Example 23, the subject matter of any one or more of Examples 19-22 optionally include, wherein comprising generate an alert or a recommendation of apnea evaluation or treatment in response to the determined apnea risk exceeding a risk threshold.

The systems, devices, and methods discussed in this document may improve the medical technology of automated device-based sleep apnea risk stratification. According to some examples, the bradycardia burden-based sleep apnea stratification can be implemented in an ambulatory (e.g., wearable or implantable) device with a simple yet highly efficient bradycardia event or sinus pause detector. Such ambulatory device provides convenient in-home sleep apnea risk assessment, and can be used to screen patients and identify those with elevated sleep apnea risk who may be recommended for further confirmatory testing such as a sleep study. As the sleep apnea risk is based on heart rate and rhythm detection, lower cost or less obtrusive systems, apparatus, and methods may be used. For example, because the system or device does not require direct or indirect respiration sensing, the system complexity and implementation cost may be reduced. It may particularly be beneficial for patients at early stage of sleep apnea or those constrained by accessibility, scheduling, affordability, or various contraindications or sleep studies.

This Overview is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIGS. 2A-2B are graphs illustrating examples of cardiac signal waveforms representative of ventricular depolarization that can be used to determine a bradycardia burden.

FIG. 3 illustrates generally an example of a sleep apnea stratification system that can determine a patient's risk of sleep apnea based at least on a bradycardia burden.

FIG. 4 is a graph illustrating an example of detecting sinus pause (a type of bradycardia) using a trend of R-wave amplitude.

FIG. 5 is a flowchart illustrating an example of monitoring a subject for risk of sleep apnea based at least on a bradycardia burden.

FIG. 6 illustrates generally a block diagram of an example machine upon which any one or more of the techniques (e.g., methodologies) discussed herein may perform.

### DETAILED DESCRIPTION

Disclosed herein are systems, devices, and methods for monitoring a subject for sleep apnea risk based on a bradycardia burden. In accordance with an embodiment, a sleep apnea monitoring system comprises a risk stratification circuit configured to detect bradycardia using cardiac information of the subject during a monitoring period, determine a bradycardia burden representing accumulated time or relative time of the subject being in the detected bradycardia over the monitoring period, and determine an apnea risk based on the bradycardia burden. An output unit can provide the apnea risk to a user, or to a process such as confirmatory detection of sleep apnea.

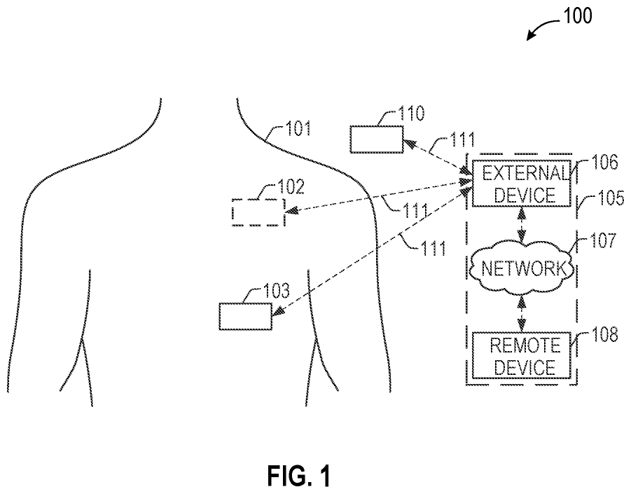

FIG. 1 illustrates an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more ambulatory medical devices, an external system 105, and a communication link 111 providing for communication between the one or more ambulatory medical devices and the external system 105. The one or more ambulatory medical devices can include an implantable medical device (IMD) 102, a wearable medical device (WMD) 103, or one or more other implantable, leadless, subcutaneous, external, wearable, or ambulatory medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 can include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 can include a monitor implanted, for example, subcutaneously in the chest of patient 101, the IMD 102 including a housing containing circuitry and, in certain examples, one or more sensors, such as a temperature sensor, etc.

The IMD 102 can include an assessment circuit configured to detect or determine specific physiologic information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the IMD 102 or one or more of the other ambulatory medical devices, etc. In some examples, therapy can include cardiac resynchronization therapy for rectifying dyssynchrony and improving cardiac function in heart failure patients. In other examples, the IMD 102 can include a drug delivery system, such as a drug infusion pump to deliver drugs to the patient for managing arrhythmias or complications from arrhythmias, hypertension, or one or more other physiologic conditions. In other examples, the IMD 102 can include one or more electrodes configured to stimulate the nervous system of the patient or to provide stimulation to the muscles of the patient airway, etc.

The WMD 103 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 105 can include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This can include, for example, programming the IMD 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information can include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 can include a medical device programmer. The remote device 108 can be configured to evaluate collected patient or patient information and provide alert notifications, among other possible functions. In an example, the remote device 108 can include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected physiological event can be prioritized using a similarity metric between the physiological data associated with the detected physiological event to physiological data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiological events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

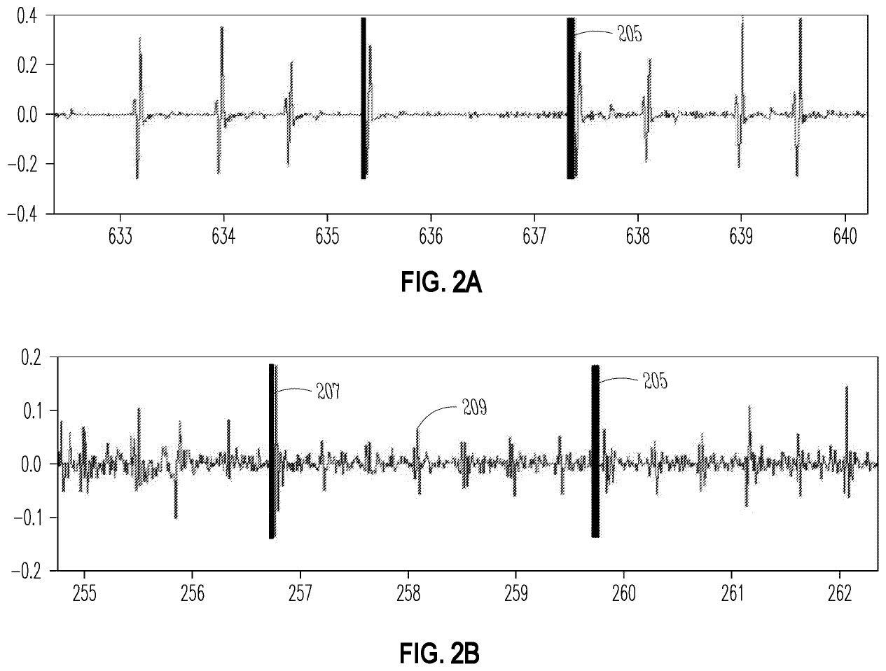

FIGS. 2A-2B are graphs of example cardiac signal waveforms representative of ventricular depolarization. FIG. 2A shows a true episode of sinus pause, characterized by temporary cessation of sinus node discharges and electrocardiographically represented by an absence of P waves (atrial depolarization) and associated QRS complex (ventricular depolarization) in an electrocardiogram (ECG). To detect a true sinus pause, the cardiac signal may be pre-processed (e.g., filtered to attenuate noise), and ventricular depolarization can be detected such as based on a comparison of signal intensity of the R wave or the QRS complex (e.g., amplitude or signal power) to a threshold. A sinus pause episode can be identified when a delay in ventricular depolarization from an immediate previous ventricular depolarization exceeds a specified pause threshold. Perschbacher et al. U.S. patent application Ser. No. 15/697,756 entitled “BRADY PAUSE DETECTION FOR IMPLANTABLE CARDIAC MONITORS,” describes techniques of detecting sinus pause (also referred to as brady pause) from cardiac signals, the disclosure of which is incorporated by reference herein in its entirety. In the example illustrated in FIG. 2A, the pause threshold for the time duration between R-waves for detecting a sinus pause is set to two seconds. The graph includes a pause marker 205 to indicate the sinus pause. As shown in FIG. 2A, the signal waveform is essentially a flat line between the last R-wave before the pause and the marker. FIG. 2B shows a false sinus pause. In this example, the threshold for the duration between R-waves for detection of sinus pause is set to three seconds. The pause marker 205 indicates when sinus pause was incorrectly declared. The large R-wave 207 before the pause marker raised the dynamic detection threshold. The subsequent R-waves of smaller amplitude 209 are missed due to the latency in the adjustment of the dynamic threshold causing the detection of a false sinus pause.

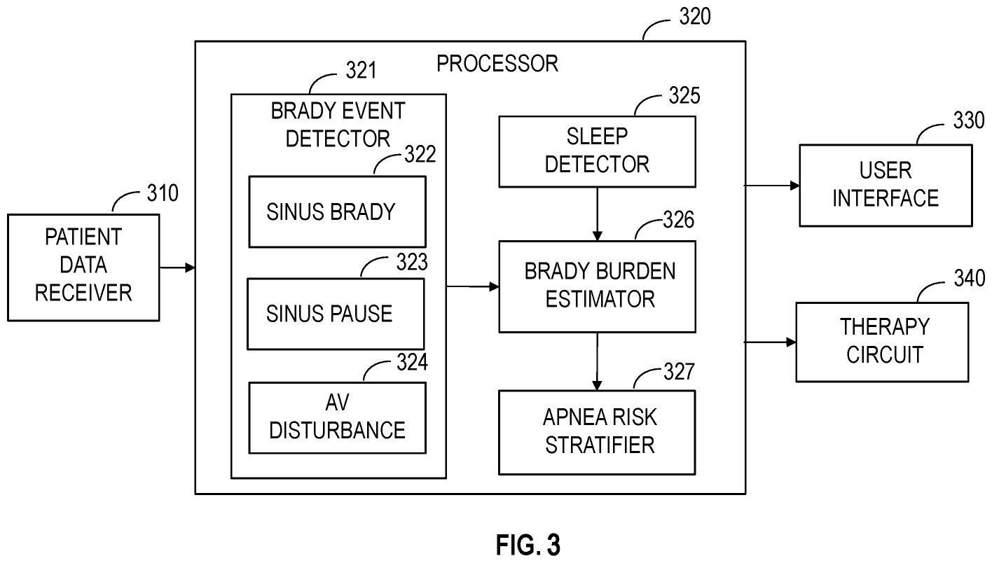

FIG. 3 illustrates generally an example of a sleep apnea stratification system 300 that can determine a patient's risk of sleep apnea based at least on a bradycardia burden indicating time spent in bradycardia over a monitoring period. Depending on the sleep apnea risk stratification, the patient may be recommended for further assessment, such as confirmatory detection of sleep apnea in a sleep study, or treatment if necessary. Portions of the sleep apnea stratification system 300 may be implemented in one or more of the IMD 102, the WMD 103, or at least a portion of the external system 105.

The sleep apnea stratification system 300 may include one or more of a patient data receiver 310, a processor 320, and a user interface 330. The sleep apnea stratification system 300 may include an optional therapy circuit 340. The patient data receiver 310 may receive physiological information from the patient. In an example, the patient data receiver 310 may include circuitry to sense a physiologic signal from the patient via one or more implantable, wearable, or otherwise ambulatory sensors or electrodes associated with the patient. Examples of the physiologic signal may include cardiac information such as surface electrocardiography (ECG), subcutaneous ECG, or intracardiac electrogram (EGM), thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal such as sensed by an ambulatory accelerometer or acoustic sensors, physiologic response to activity, apnea hypopnea index, one or more respiration signals such as a respiration rate signal or a tidal volume signal, brain natriuretic peptide (BNP), blood panel, sodium and potassium levels, glucose level and other biomarkers and bio-chemical markers, among others. The patient data receiver 310 may include one or more other sub-circuits to digitize, filter, or perform other signal conditioning operations on the received physiologic signal. In some examples, The patient data receiver 310 may receive physiological information from a storage device such as an electronic medical record system that stores physiological information collected from the patient.

The processor 320, coupled to the patient data receiver 310, may determine a sleep apnea risk using the physiological information such as cardiac information received from the patient data receiver 310. The processor 320 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The processor 320 may include circuit sets comprising one or more other circuits or sub-circuits, including a brady event detector 321, an sleep detector 325, a brady burden estimator 326, and an apnea risk stratifier 327. These circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

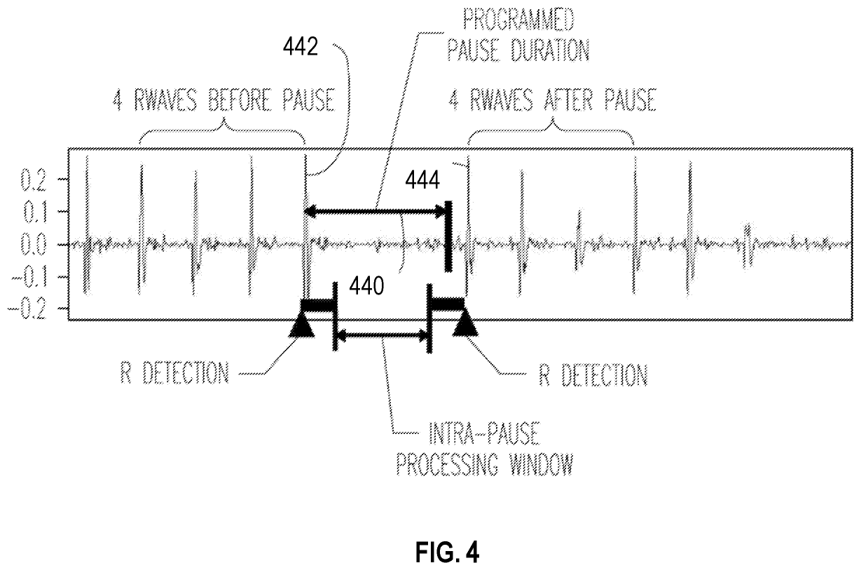

The brady event detector 321 can detect bradycardia using the physiological information such as cardiac information received from the patient data receiver 310. In an example, the bradycardia may be detected during a specific monitoring period. The detected bradycardia may include several types of heart rhythms characterized by slow heart rates. By way of example and not limitation, such bradycardia events may include sinus brady 322, sinus pause 323, and atrioventricular (AV) disturbance 324. Other bradycardia events may include sick sinus syndrome associated with disease or malfunction of the sinus node, tachy-brady syndrome where heart sometimes beats too quickly (tachy) and sometimes beats too slowly (brady). The brady event detector 321 can pre-process a cardiac signal (e.g., ECG or EGM) such as filtering out or attenuating noise or interferences, and detect cardiac activities such as atrial depolarization (e.g., P waves on an ECG, or on intracardiac atrial EGM) or ventricular depolarization based on signal amplitude or signal morphology. Timings of the detected atrial or ventricular depolarization can be used to determine one or more of sinus brady 322, sinus pause 323, or AV disturbance 324. For example, the sinus brady 322 may be detected if the heart rate falls below a threshold, or equivalently if the cardiac cycle length (the time interval between consecutive heart beats) exceeds a threshold, during a monitoring period. The sinus pause 323 can be identified when a delay in ventricular depolarization from an immediate previous ventricular depolarization exceeds a specified pause threshold. Perschbacher et al. U.S. patent application Ser. No. 15/697,756 entitled “BRADY PAUSE DETECTION FOR IMPLANTABLE CARDIAC MONITORS,” describes various embodiments of a sinus pause detector, which is incorporated by reference herein in its entirety. An example technique of detecting sinus pause is described below with reference to FIG. 4. The AV disturbance 324 represents various degrees or patterns of AV block, and can be detected when an AV interval, measured from atrial depolarization to ventricular depolarization (e.g., P wave to R wave interval, or atrial EGM peak to ventricular EGM peak) within the same cardiac cycle exceeds an AV interval threshold, or when the AV interval variability over multiple cardiac cycles exceeds a variability threshold. Perschbacher et al. U.S. Pat. No. 11,207,528 entitled “ATRIOVENTRICULAR CONDUCTION GUIDED HIS-BUNDLE PACING” describes techniques of detecting AV conduction disturbance using cardiac signals, the disclosure of which is incorporated by reference herein in its entirety.

The brady burden estimator 326 can estimate a bradycardia burden representing accumulated time or relative time of the subject being in the detected bradycardia (e.g., one or more of sinus brady 322, sinus pause 323, or AV disturbance 324) over a specific monitoring period. In an example, the bradycardia burden can be calculated as the proportion of time, expressed as a percentage, the subject is in the bradycardia during the monitoring period. In another example, the bradycardia burden can be determined as the duration of the longest bradycardia episode during the monitoring period. In yet another example, the bradycardia burden can be determined as a count of the bradycardia episodes detected during the monitoring period.

In some examples, the brady burden estimator 326 can estimate the bradycardia burden using a selected subset of the detected bradycardia events each satisfying a specific requirement. Examples of such requirement can include a heart rate range or threshold, or a pause duration range of threshold. For example, the bradycardia burden can be determined based on the number of sinus pause events of at least 5-second long, or the number of brady events with an average heart rate of no greater than 30 beats per minute (bpm), that have been detected during the monitoring period.

The monitoring period during which the bradycardia burden is determined can be a predetermined time period, such as one day (24 hours), a week, two weeks, or one month, among others. Alternatively, the monitoring period can be a specific time of a day, such as nighttime. As previously described, nocturnal bradycardia events (e.g., sinus pauses during the nighttime) may be associated with incidents of sleep apnea. In an example, the monitoring period can be determined based on a patient state or health condition, such as a sleep/awake state. The processor 320 may include a sleep detector 325 that can automatically detect a sleep or awake state using one or more sensors such as an accelerometer, a piezoelectric sensor, biopotential electrodes, or other physiologic sensors configured to detect posture, a change in posture, physical activity or exertions levels, respiration, heart rate, electroencephalograms, or other signals indicative of a sleep state, an awake state, or transition between sleep and awake states. Additionally or alternatively, the sleep detector 325 may receive information about sleep and awake state from an end-user such as via a user-interface, or via a smart device (e.g., a smart wearable device) capable of detecting sleep and awake states. The brady burden estimator 326 can estimate the bradycardia burden using bradycardia events detected when the subject is asleep.

The apnea risk stratifier 327 can determine an apnea risk based on the bradycardia burden. In an example, the apnea risk can be determined based on a comparison of the bradycardia burden to a threshold burden. In some examples, the apnea risk stratifier 327 can track the bradycardia burden over time, such as by generating a trend of daily or weekly bradycardia burden, and the apnea risk can be estimated based on an increase trend of bradycardia burden. In some examples, bradycardia burden may be respectively determined for distinct bradycardia events, such as a burden of sinus brady, a burden of sinus pause, or a burden of AV disturbance. The apnea risk stratifier 327 can determine an apnea risk using a linear or a nonlinear combination of event-specific burdens respectively determined for different bradycardia events. In an example, the apnea risk can be determined using a weighted combination of the event-specific burdens each scaled by respective weight factors.

The apnea risk can be represented by a numerical risk score directly proportional to the determined bradycardia burden, such that a higher bradycardia burden corresponds to a higher sleep apnea risk. A categorical apnea risk can be determined by comparing the numerical apnea risk score to one or more thresholds or ranges of scores.

The user interface 330 may include an input device and an output device. In an example, at least a portion of the user interface unit 250 may be implemented in the external system 105. The input device may receive a user's programming input, such as parameters for cardiac signal processing, bradycardia event detection, sleep detection, or brady burden estimation. The input device may include a keyboard, on-screen keyboard, mouse, trackball, touchpad, touch-screen, or other pointing or navigating devices. The input device may enable a system user to program the parameters used for sensing the physiologic signals, detecting the arrhythmias, and generating alerts, among others. The output device may generate a human-perceptible presentation of. The output device may include a display for displaying cardiac signals used for apnea risk assessment, intermediate measurements or computations such as bradycardia burden, and the apnea risk. The output unit may include a printer for printing hard copies of the detection information. The information may be presented in a table, a chart, a diagram, or any other types of textual, tabular, or graphical presentation formats. The presentation of the output information may include audio or other media format. The output unit may generate an alert to the system user or a recommendation of apnea evaluation or treatment in response to the determined apnea risk exceeding a risk threshold. In some examples, the output unit may provide a control signal to a respiration-based apnea detector to trigger sleep apnea detection. In an example, the respiration-based sleep apnea detector can be included in an ambulatory medical device such as the IMD 102 or the WMD 103. The respiration-based sleep apnea detector can use respiration parameters, transthoracic impedance, acoustic signals indicative of respiration, or other respiration surrogates to detect sleep apnea.

The optional therapy circuit 340 may be configured to deliver a therapy to the patient based on the estimated brady burden or the apnea risk. Examples of the therapy may include electrostimulation therapy delivered to the heart, a nerve tissue, other target tissues, a cardioversion therapy, a defibrillation therapy, or drug therapy. In some examples, the therapy circuit 340 may modify an existing therapy, such as adjust a stimulation parameter or drug dosage. The therapy can help reduce brady burden and lower the apnea risk.

FIG. 4 is a graph illustrating an example of detecting sinus pause using a trend of R-wave amplitude such as measured from patient's cardiac information such as a ECG signal. The sinus pause detection technique described herein can be used by the brady event detector 321 to detect sinus pause 323 from a cardiac signal received by the patient data receiver 310. The sinus pause is evident in the candidate pause signal by the gap in R-waves. An intra-pause duration 440 is timed in the candidate pause signal. The timing of the intra-pause duration begins a specified time after the last R-wave sensed 442 before the detected candidate pause episode. In some examples, the intra-pause duration begins after a refractory period is timed for the last R-wave sensed 442. A refractory period refers to the period of time that sense amplifiers of a CFM are blanked or disabled to be unresponsive to intrinsic signal of the heart. The pause refractory may be a programmable pause refractory specifically used for brady pause detection.

The pause detection circuit may determine a pre-pause central tendency value (e.g., an average value or a mean value) of the amplitude of a specified number of R-waves identified prior to the candidate pause episode. In the example as shown in FIG. 4, four R-waves are used in determining the pre-pause central tendency value.

The pause detection circuit may also calculate an intra-pause threshold of amplitude of signal samples during the intra-pause duration. These signal samples would be included after the last R-wave sensed 442 and before the first R-wave 444 sensed after the detected candidate pause. In some examples, the intra-pause threshold is calculated according to the amplitude of a specified fraction of the signal samples during the intra-pause duration. For instance, the intra-pause threshold may be calculated as a percentile amplitude value (e.g., the 95^{th}, the 98^{th} percentile value, or even the 100^{th} percentile value) of the signal samples in the intra-pause duration window.

A criterion for determining a false brady pause may include the pre-pause central tendency value and the intra-pause threshold in a signal-to-noise metric. For instance, the pause detection circuit may calculate a ratio that includes the pre-pause central tendency value and the intra-pause threshold. The pause detection circuit discards the candidate pause episode or stores the candidate pause episode as a bradycardia pause episode according to the pre-pause central tendency value and the intra-pause threshold. For instance, the pause detection may discard the candidate pause episode when a calculated ratio of the pre-pause central tendency value to the intra-pause threshold is less than a specified ratio threshold value.

The pause detection circuit may determine a post-pause central tendency value (or look-ahead central tendency value) of the amplitude of a specified number of R-waves identified after the candidate pause episode. In the example shown in FIG. 4, four R-waves are used in determining the post-pause central tendency value. A signal-to-noise metric to detect a false brady pause may include the post-pause central tendency value and the intra-pause threshold (e.g., a ratio including the pre-pause central tendency value and the intra-pause threshold). The pause detection circuit discards the candidate pause episode or stores the candidate pause episode as a bradycardia pause episode according to the post-pause central tendency value and the intra-pause threshold. For instance, the pause detection may discard the candidate pause episode when a calculated ratio of the post-pause central tendency value to the intra-pause threshold is less than a specified ratio threshold value.

In some examples, the pause detection circuit discards the candidate pause episode or stores the candidate pause episode as a bradycardia pause episode according to the pre-pause central tendency value, the post-pause central tendency value and the intra-pause threshold (e.g., a pre-pause ratio and a post-pause ratio). In some examples, the pause detection circuit rejects the candidate pause episode if a specified number of R-waves are not detected during a specified post-pause duration. For instance, the pause detection circuits may reject the candidate pause episode if four R-waves are not detected within eight seconds after the last R-wave sensed 442 before the detected candidate pause episode.

Other signal-to-noise metrics may be used by the pause detection circuit to determine brady pause or false brady pause. In some examples, the pause detection circuit calculates the pre-pause central tendency value of the R-wave amplitude and calculates the intra-pause threshold for the intra-pause duration using the pre-pause central tendency value (e.g., as a fraction of the pre-pause central tendency value). The pause detection then identifies signal samples during the intra-pause duration that exceed the calculated intra-pause threshold.

When the number of identified signal samples exceeds a specified threshold number of signal samples (e.g., 2% of the number of signal samples included in the intra-pause duration), the pause detection circuit discards the candidate pause episode. This process can be viewed as a shortcut method similar to using a signal-to-noise ratio of the pre-pause central tendency value to the intra-pause threshold. As soon as the number of signal samples exceeds the specified threshold number (e.g., the 2%) it is known that the ratio of the pre-pause central tendency value to the specified amplitude percentile (e.g., the 98^{th} percentile) will be less than the specified threshold. The candidate pause episode may be discarded when the number of signal samples exceeds the specified threshold number.

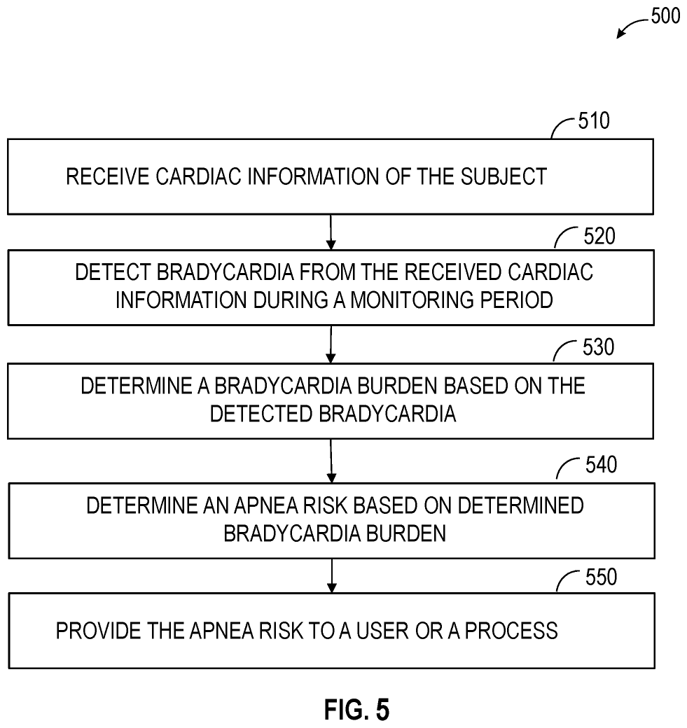

FIG. 5 is a flowchart illustrating an example of a method 500 of monitoring a subject for risk of sleep apnea. The sleep apnea risk can be estimated based on a bradycardia burden. The method 500 may be implemented in and executed using the sleep apnea stratification system 300.

The method 500 begins at step 510, where cardiac information of a subject may be received such as using the patient data receiver 310. The cardiac information may include ECG or EGM, or signals indicative of cardiac mechanical activity, such as pressure, impedance, heart sounds, or respiration signals. The sensed cardiac information may be pre-processed, including amplification, digitization, filtering, or other signal conditioning operations. In some examples, patient cardiac information may be sensed and stored in a storage device, such as an electronic medical record system, and retrieved for use upon a user or a system request.

At 520, bradycardia may be detected from the received cardiac information during a monitoring period, such as by using the brady event detector 321. The detected bradycardia may include several types of heart rhythms characterized by slow heart rates, such as sinus brady, sinus pause, atrioventricular (AV) disturbance, among others. Amplitude or morphology, among other signal metrics, may be extracted from the received cardiac signal and used to detect various types of bradycardia events, as described above with reference to FIG. 4.

At 530, a bradycardia burden may be determined based on the detected bradycardia. The bradycardia burden can represent accumulated time or relative time of the subject being in the bradycardia during a monitoring period. In an example, the bradycardia burden can be calculated as the proportion (e.g., percentage) of time the subject is in the bradycardia during the monitoring period. In another example, the bradycardia burden can be determined as the duration of the longest bradycardia episode during the monitoring period. In yet another example, the bradycardia burden can be determined as a count of bradycardia episodes detected during the monitoring period. In some examples, the bradycardia burden may be estimated using a subset of the detected bradycardia events each satisfying a specific heart rate or duration requirement. For example, the bradycardia burden can be determined based on the number of sinus pause episodes of at least 5-second long, or the number of brady events with an average heart rate of no greater than 30 beats per minute, that have been detected during the monitoring period.

The monitoring period during which the bradycardia burden is determined can be a predetermined time period, such as one day (24 hours), a week, two weeks, or one month, among others. Alternatively, the monitoring period can be a specific time of a day, such as nighttime. In some examples, the monitoring period can be determined based on a patient state or health status, such as a sleep/awake state. In an example, sleep may be automatically detected such as using the sleep detector 325, and the bradycardia burden can be estimated using bradycardia events detected when the subject is asleep.

At 540, an apnea risk may be determined based on the bradycardia burden, such as using the apnea risk stratifier 327. The apnea risk can be determined based on a comparison of the bradycardia burden to a threshold burden. In an example, the bradycardia burden can be trended over time, and the apnea risk can be estimated based on an increase trend of bradycardia burden. In some examples, the bradycardia burden may be respectively determined for distinct bradycardia events, such as a burden of sinus brady, a burden of sinus pause, or a burden of AV disturbance. An apnea risk can be determined using a linear or a nonlinear combination of the event-specific burdens.

At 550, the apnea risk can be provided to a user or process, such as via an output device of the user interface 330. In an example, the apnea risk and the bradycardia burden may be displayed on a display, optionally along with the cardiac signals from which the bradycardia events were detected. In various examples, alerts, alarms, emergency calls, or other forms of warnings may be generated to signal the user about the apnea risk, particularly when the apnea risk exceeds a threshold. In some examples, a recommendation may be generated and provided to a user. The recommendation of further apnea evaluation or treatment may be provided to the user. In some examples, in response to the determined apnea risk exceeding a risk threshold, a control signal may be generated to trigger a respiration-based apnea detection.

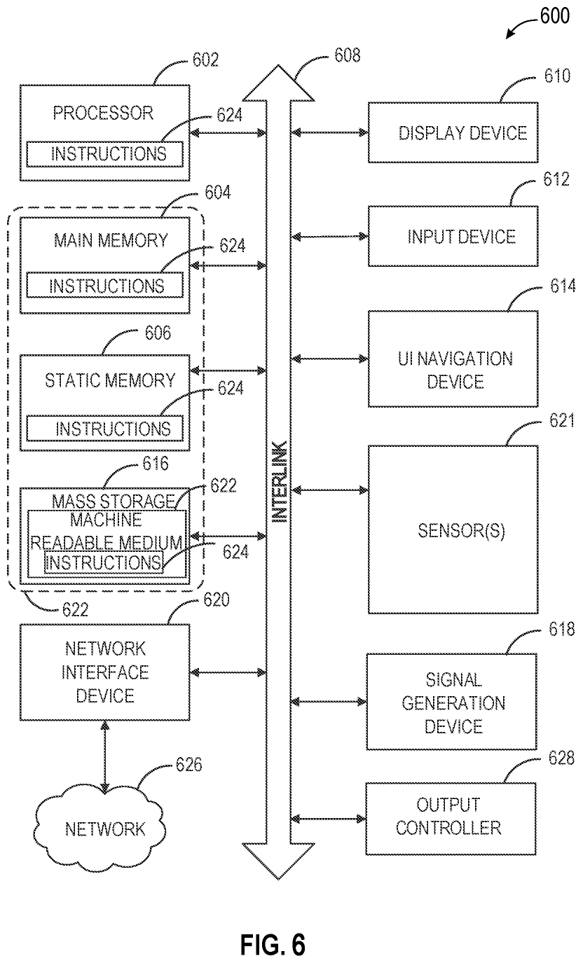

FIG. 6 illustrates generally a block diagram of an example machine 600 upon which any one or more of the techniques (e.g., methodologies) discussed herein may perform. Portions of this description may apply to the computing framework of various portions of the patient management system 100 or of the sleep apnea stratification system 300.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine-readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine-readable media.

While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802. 11 family of standards known as WiFi®, IEEE 802. 16 family of standards known as WiMax®), IEEE 802. 15. 4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for monitoring a subject for risk of sleep apnea, the system comprising:
a sleep apnea risk stratification circuit configured to:
receive cardiac information of the subject;
detect bradycardia from the received cardiac information during a monitoring period;
determine a bradycardia burden based on the detected bradycardia, the bradycardia burden representing accumulated time or relative time of the subject being in the bradycardia during the monitoring period; and
determine an apnea risk based on the bradycardia burden; and
an output unit configured to provide the determined apnea risk to a user or a process.

2. The system of claim 1, comprising an ambulatory medical device configured to collect the cardiac information including a surface or subcutaneous electrocardiogram from the subject, the ambulatory medical device including the sleep apnea risk stratification circuit.

3. The system of claim 1, wherein to detect the bradycardia includes to determine a duration of a bradycardia episode, the bradycardia episode including one or more of a sinus pause episode, a sinus bradycardia episode, or an atrioventricular (AV) disturbance or AV block episode.

4. The system of claim 1, wherein the sleep apnea risk stratification circuit is configured to determine the bradycardia burden based on the bradycardia detected during a nighttime monitoring period.

5. The system of claim 1, wherein the sleep apnea risk stratification circuit is configured to determine the bradycardia burden based on the bradycardia detected during a time period when the subject is asleep.

6. The system of claim 5, comprising a sleep detector configured to detect a sleep or awake state of the subject,
wherein the sleep apnea risk stratification circuit is configured to determine the bradycardia burden based on the bradycardia detected when the detected sleep or awake state indicates that the subject is asleep.

7. The system of claim 1, wherein the sleep apnea risk stratification circuit is configured to detect one or more bradycardia episodes each satisfying a rate or duration requirement, and to determine the bradycardia burden based on a count of the detected one or more bradycardia episodes that satisfy the rate or duration requirement during the monitoring period.

8. The system of claim 7, wherein the one or more bradycardia episodes include one or more sinus pause episodes each lasting at least a threshold duration.

9. The system of claim 7, wherein the one or more bradycardia episodes include one or more sinus bradycardia episodes each having an average heart rate below a threshold heart rate.

10. The system of claim 1, wherein the sleep apnea risk stratification circuit is configured to trend the bradycardia burden over time, and to determine the apnea risk based on the trended bradycardia burden.

11. The system of claim 1, wherein the output unit is configured to generate an alert or a recommendation of apnea evaluation or treatment in response to the determined apnea risk exceeding a risk threshold.

12. The system of claim 1, wherein the output unit is configured to generate a control signal to a respiration-based apnea detector to trigger sleep apnea detection in response to the determined apnea risk exceeding a risk threshold.

13. A method of monitoring a subject for risk of sleep apnea, the method comprising:
receiving cardiac information of the subject;
detecting bradycardia from the received cardiac information during a monitoring period;
determining a bradycardia burden based on the detected bradycardia, the bradycardia burden representing accumulated time or relative time of the subject being in the bradycardia during a monitoring period; and
determining an apnea risk based on the bradycardia burden using a sleep apnea risk stratification circuit; and
providing the determined apnea risk to a user or a process.

14. The method of claim 13, wherein determining the bradycardia burden is based on the bradycardia detected during a nighttime monitoring period.

15. The method of claim 13, wherein determining the bradycardia burden is based on the bradycardia detected during a monitoring period when the subject is asleep.

16. The method of claim 13, wherein:
detecting the bradycardia incudes detecting one or more bradycardia episodes each satisfying a rate or duration requirement,
determining the bradycardia burden is based on a count of the detected one or more bradycardia episodes during the monitoring period.

17. The method of claim 16, wherein the one or more bradycardia episodes include one or more sinus pause episodes each lasting at least a threshold duration.

18. The method of claim 16, wherein the one or more bradycardia episodes include one or more sinus bradycardia episodes each having an average heart rate below a threshold heart rate.

19. The method of claim 16, comprising generating a bradycardia burden trend over time, wherein determining the apnea risk is based on the bradycardia burden trend.

20. The method of claim 16, wherein comprising generate an alert or a recommendation of apnea evaluation or treatment in response to the determined apnea risk exceeding a risk threshold.
